(19) United States
(12) Patent Application Publication
(10) Pub. No.: US 2003/0082228 A1
(43) Pub. Date: May 1, 2003

(54) Anti-angiogenic therapy using liposome-encapsulated chemotherapeutic agents

(75) Inventors: Clay Flowers, Surrey (CA); David Saltman, Vancouver (CA); Patrick M.S. Tam, Vancouver (CA); Clive T.R. Burge, Brentwood Bay (CA); Troy O. Hasrasym, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Inex Pharmaceuticals Corporation, Burnaby (CA)

(21) Appl. No.: 10/143,545

(22) Filed: May 9, 2002

Related U.S. Application Data
(60) Provisional application No. 60/289,935, filed on May 9, 2001.

(51) Int. Cl. A61K 9/127; A61K 31/4745

(52) U.S. Cl. 424/450; 514/283

(57) ABSTRACT

The present invention provides methods and compositions for the treatment and prevention of any of a large number of diseases and conditions with an angiogenic component, e.g., cancer. The present invention is based upon the discovery that liposome-encapsulated chemotherapeutic agents, such as alkaloids (e.g., vinca alkaloids such as vincristine), are surprisingly effective at treating such diseases or conditions when administered at a higher frequency than those used with conventional administration strategies. Such methods can be used to treat diseases such as cancer even when the cancer comprises cells that are resistant to the chemotherapeutic alkaloid. The liposome encapsulation of the chemotherapeutic agents, e.g., alkaloids, imparts dramatic improvements in the stability, biodistribution, and delivery of the agents, thereby allowing more efficacious and convenient administration to a patient with any of the herein-described diseases or conditions.

### CROSS-REFERENCE TO RELATED APPLICATION

The present application claims priority to U.S. Provisional Patent Application No. 60/289,935, filed May 9, 2001, the teachings of which are incorporated herein by reference for all purposes.

### FIELD OF THE INVENTION

The present invention provides novel methods and compositions for the treatment and prevention of diseases and conditions having an angiogenic component, such as cancer.

### BACKGROUND OF THE INVENTION

Conventional chemotherapeutic regimens focus on killing as many rapidly dividing cancer cells as possible while maintaining an acceptable collateral toxicity profile in the patients receiving the treatment. Such regimens thus typically involve the periodic administration of a “maximum tolerated dose” of a drug, followed by an extended treatment-free period to permit recovery of the patient, e.g., regrowth of rapidly growing hematopoietic progenitors. Such high levels of administration can produce various side effects, including neurotoxicity and other damage to non-cancerous cells in the patient. In addition, such conventional treatments often fail to completely eradicate the cancer, and are thus commonly followed by relapses, sometimes involving cells that are resistant to the compound. Clearly, new, effective, and safe approaches for treating cancer are needed.

One recently-developed strategy for treating cancer targets angiogenesis, the process by which tumors induce the formation of new blood vessels in order to divert the host blood supply towards itself. Because tumor growth is dependent on a constant supply of blood, the inhibition of angiogenesis prevents the growth and maintenance of existing tumors, as well as the appearance of new tumors. Indeed, anti-angiogenic agents have been shown to reduce established tumors in preclinical models, and can prevent the growth of tumors beyond 1-2 mm³. A number of compounds have been shown to possess anti-angiogenic activity, including platelet factor-4, angiostatin, endostatin, interferon alpha or beta, and vasostatin. For reviews, see, e.g., Griffioen, et al., Pharmacol. Rev., 52:237-68 (2000); and Rosen, Oncologist, 5 Suppl. 1:20-7 (2000). In addition, many traditional chemotherapeutic agents have anti-angiogenic activity due to their effect on dividing endothelial cells, although the traditional, high level administration of these agents has usually masked this anti-angiogenic effect in vivo.

A number of diseases other than cancer also have an angiogenic component, including age-related macular degeneration, diabetic retinopathy, rubeotic glaucoma, interstitial keratitis, retinopathy of prematurity, corneal graft failure, psoriasis, atherosclerosis, restenosis, chronic inflammation, rheumatoid arthritis, vasculopathies including hemangiomas and systemic vasculitis. Such diseases can also be treated or prevented in a patient by the inhibition of angiogenesis.

Alkaloids isolated from the periwinkle plant (Vinca rosea), called “vinca alkaloids,” have proven effective for the treatment of many types of lymphomas, leukemias, and other cancers. One such vinca alkaloid, vincristine, is included in the common chemotherapeutic formulation CHOP. Vincristine, which depolymerizes microtubules and thereby inhibits cell proliferation, is administered in its free form in CHOP. Liposome-encapsulated vincristine has been reported, e.g., in U.S. Pat. Nos. 5,741,516 and 5,714,163, which discuss the use of vincristine encapsulated in phosphatidylcholine, distearoylphosphatidylcholine, or sphingomyelin, in addition to cholesterol.

Lipid-encapsulated drug formulations provide numerous advantages over traditional drug-delivery methods. For example, some lipid-based formulations provide longer half-lives in vivo, superior tissue targeting, and decreased toxicity. Numerous methods have been described for the formulation and use of lipid-based drug delivery vehicles (see, e.g., U.S. Pat. No. 5,741,516; and Chonn, et al., Curr. Opin. Biotechnol., 6:698-708 (1995)).

Accordingly, there remains a great need in the art for new methods for treating cancer and other angiogenesis-related diseases and conditions. The present invention addresses this and other needs.

### SUMMARY OF THE INVENTION

The present invention provides methods for the treatment and prevention of any of a large number of diseases and conditions having an angiogenic component. This invention is based upon the surprising discovery that the frequent administration of a liposome-encapsulated chemotherapeutic agent, such as a chemotherapeutic alkaloid, to a patient inhibits the angiogenesis that is associated with and required for the progression or appearance of the disease or condition. Often, the present methods are administered to a patient for maintenance therapy, e.g., following a primary treatment for the disease, and are thus administered for a relatively long period of time, in some cases indefinitely, in order to prevent the recurrence of the disease. In contrast to conventional therapies, which are aimed to deliver the maximum tolerated dose over a short period of time, followed by a “rest” period during which the body can recover and the compound is cleared from the body, the present methods are directed to providing a long term, sustainable, level of the compound. As such, the dosage forms used according to the present invention are often lower than those used with conventional strategies.

In one aspect, therefore, the present invention provides a method of treating or preventing a disease or condition having an angiogenic component in a mammal, the method comprising administering to the mammal a pharmaceutical composition comprising a liposome-encapsulated chemotherapeutic agent, such as a chemotherapeutic alkaloid, wherein the pharmaceutical composition is administered to the mammal at an average frequency of at least once every 7 days for a total period of at least 6 weeks.

In one embodiment, the disease or condition is cancer (e.g., prostate, lung, breast, colon, kidney, stomach, bladder, or ovarian cancer, multiple myeloma, etc.). In another embodiment, the chemotherapeutic agent is a vinca alkaloid. In another embodiment, the vinca alkaloid is vincristine. In another embodiment, the vincristine is administered to the mammal at a dosage that is a fraction (e.g., half) of the maximum tolerated dose or the normal clinical dose. In another embodiment, the vincristine is administered to the mammal at a dosage of less than 0.5 mg/m². In another embodiment, the vinca alkaloid is vinorelbine or vinblastine. In another embodiment, the chemotherapeutic agent is camptothecin or a camptothecin analog. In another embodiment, the camptothecin is topotecan. In another embodiment, the composition is administered to the mammal for a total period of at least 10 weeks. In another embodiment, the method further comprises co-administering an angiogenesis inhibitor to the mammal. In another embodiment, the angiogenesis inhibitor includes, but is not limited to, thrombospondin, internal fragments of thrombospondin, angiostatin, endostatin, vasostatin, vascular endothelial growth factor inhibitor (VEGI), fragment of platelet factor 4 (PP4), derivative of prolactin, restin, proliferin-related protein (PRP), SPARC cleavage product, osteopontin cleavage product, interferon α, interferon β, meth 1, meth I, angiopoietin-2, anti-thrombin III fragment, COL-3, squalamine, combretastatin, PTK787/ZK2284, CAI, PIK787/2K22584, CGS-27023A, TNP-470, thalidomide, SU5416, vitaxin, IL-12, EMD121974, marimastat, AG3340, neovastat/AE941, anti-VEGF Ab, and IM862.

In another embodiment, the liposome comprises sphingomyelin. In another embodiment, the liposome further comprises cholesterol. In another embodiment, the liposome comprises a PEG-lipid. In another embodiment, the liposome comprises an ATTA-lipid. In another embodiment, the pharmaceutical composition is administered to the patient following a primary cancer treatment, and the method is used to delay or prevent relapse of the cancer in the patient. In another embodiment, the pharmaceutical composition is administered to the patient following a primary cancer treatment, and the method is used to prevent metastasis of the cancer in the patient. In one such embodiment, the primary cancer is colorectal cancer, and the method is used to prevent metastasis of the cancer to the liver.

In another embodiment, the cancer comprises cancer cells that are resistant to the chemotherapeutic agent, such as the chemotherapeutic alkaloid. In another embodiment, the method further comprises co-administering to the mammal an oligonucleotide agent. In another embodiment, the disease includes, but is not limited to, age-related macular degeneration, diabetic retinopathy, rubeotic glaucoma, interstitial keratitis, retinopathy of prematurity, corneal graft failure, psoriasis, atherosclerosis, restenosis, chronic inflammation, rheumatoid arthritis, vasculopathies including hemangiomas and systemic vasculitis.

In another aspect, the present invention provides a method of treating a chemotherapeutic agent (e.g., chemotherapeutic alkaloid)-resistant tumor in a mammal, the method comprising administering to the mammal a pharmaceutical composition comprising the chemotherapeutic agent, such as an alkaloid, in a liposome-encapsulated form, wherein the pharmaceutical composition is administered to the mammal at an average frequency of at least once every 7 days for a total period of at least 6 weeks. In a preferred embodiment, the pharmaceutical composition is administered to the mammal at an average frequency of at least once every 7 days for a total period of at least 7, 8, 9 or 10 weeks and, more preferably, for a total period of longer than 10 weeks. In another preferred embodiment, the pharmaceutical composition is administered to the mammal continuously for a period of several days to weeks to months (e.g., 1, 2, 3, 4, 5, 6, 7, 8, 9, 10, 11, 12 months, etc.).

In one embodiment, the chemotherapeutic alkaloid is a vinca alkaloid. In another embodiment, the vinca alkaloid is vincristine. In another embodiment, the tumor is a breast tumor.

In another aspect, the present invention provides a dosage form of liposome-encapsulated vincristine, the dosage form comprising less than the Maximum Tolerated Dose (MTD) of 2.0 mg/m² of vincristine per dose. In a presently preferred embodiment, the present invention provides a dosage form of liposome-encapsulated vincristine, the dosage form comprising less than about 0.5 mg/m² of vincristine per dose.

In one embodiment, the vincristine is present at less than about 0.2 mg/m² per dose. In one embodiment, the vincristine is present at less than about 0.1 mg/m² per dose. In another embodiment, the liposome comprises sphingomyelin. In another embodiment, the liposome further comprises cholesterol. In another embodiment, the liposome comprises a PEG-lipid. In another embodiment, the liposome comprises an ATTA-lipid.

Kits for practicing the present invention are also provided.

### DETAILED DESCRIPTION OF THE INVENTION AND PREFERRED EMBODIMENTS

### I. Introduction

The present invention provides methods and compositions for the treatment and prevention of any of a large number of diseases and conditions having an angiogenic component, e.g., cancer. The present invention is based upon the discovery that liposome-encapsulated chemotherapeutic agents, such as chemotherapeutic alkaloids (e.g., vinca alkaloids such as vincristine), are surprisingly effective at treating such diseases or conditions when administered at a higher frequency than those used with conventional administration strategies. Such methods can be used to treat diseases such as cancer even when the cancer comprises cells that are resistant to the chemotherapeutic alkaloid. The liposome encapsulation of the chemotherapeutic agents, such as chemotherapeutic alkaloids, imparts dramatic improvements in the stability, biodistribution, and delivery of the agents, thereby allowing more efficacious and convenient administration to a patient with any of the herein-described diseases or conditions.

According to the present methods, chemotherapeutic agents, such as chemotherapeutic alkaloids, are administered to patients at doses which prevent the growth and development of neovasculature at disease sites, while staying well below toxic levels of the agent, thereby permitting extended, long-term therapy, e.g., maintenance therapy, in patients. Because of the high frequency of administration, as well as the liposome encapsulation of the agents, the agents are typically continuously present at the disease site within the patient, although typically at a lower level than according to conventional dosing strategies. This constant level of agent, e.g., alkaloid, prevents the growth and division of endothelial cells associated with the disease or condition, thereby preventing angiogenesis and disease progression. For cancer, the present methods can be used to inhibit neovascularization of established tumors as well as of micrometastases.

Generally, the present methods involve administering a liposome-encapsulated agents, such as an alkaloid (e.g., vinca alkaloid such as vincristine), at a relatively high frequency, e.g., once every 1, 2, 5, 7, or more days. Typically, the methods are performed continuously over a relatively long period of time, e.g., 4, 6, 8, 10, 12, 14, 16 or more weeks.

Liposome-encapsulated alkaloids, e.g., vinca alkaloids such as vincristine, and methods of their use in the treatment of diseases such as cancer are described, e.g., in U.S. patent application Nos. 60/127,444, 60/137,194, and 09/541,436, the disclosures of which are incorporated herein in their entirety by reference.

### II. Definitions

A “chemotherapeutic alkaloid” refers to any of a large number of nitrogenous substances that are either found naturally in plants or synthesized de novo, which have potential use as a chemotherapeutic agent, i.e., use in treating cancer. Such chemotherapeutic alkaloids thus possess an activity useful for the treatment of cancer, e.g., cytotoxic, cytostatic, apoptosis-inducing, anti-mitogenic, immuno-stimulatory, or other effects. Examples of suitable alkaloids for use in the present invention include, but are not limited to, podophyllins, podophyllotoxins, camptothecins, vinca alkaloids such as vincristine, vinorelbine, vinblastine and vindesine, and derivatives of these compounds.

A mammal refers to any member of the Class Mammalia. As used herein, mammals include any of a number of experimental animals such as rodents, as well as bovines, porcines, lagomorphs, canines, felines, equines, and primates including humans. A “patient” refers to any mammal with or at risk of developing any of the diseases or conditions described herein.

A disease or condition having an “angiogenic component” refers to any disease or condition, in any mammal, of which the appearance, progression, stability, severity, or any other quality, is dependent or influenced by the formation of new blood vessels. Examples of such diseases include cancers, including cancers comprising solid tumors and blood-based cancers such as lymphomas, leukemias and multiple myeloma, as well as any of a large number of ocular diseases, vascular diseases, and chronic inflammatory disorders.

“Neoplasia,” as used herein, refers to any aberrant growth of cells, tumors, malignant effusions, warts, polyps, nonsolid tumors, cysts and other growths. A site of neoplasia can contain a variety of cell types including, but not limited to, neoplastic cells, vascular endothelia, or immune system cells, such as macrophages and leukocytes, etc.

“Cancer” in a mammal refers to any of a number of conditions caused by the abnormal, uncontrolled growth of cells. Cells capable of causing cancer, called “cancer cells,” can possess any of a number of characteristic properties such as uncontrolled proliferation, immortality, metastatic potential, rapid growth and proliferation rate, and certain typical morphological features. Often, cancer cells will be in the form of a tumor, but such cells may also exist alone within the mammal. Cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer (e.g., CA125, PAP, PSA, CEA, AFP, HCG, CA 19-9, CA 15-3, CA 27-29, LDH, NSE, and others), and detecting a genotype indicative of a cancer (e.g., TP53, ATM, etc.). However, a negative result in one or more of the above detection methods does not necessarily indicate the absence of cancer, e.g., a patient who has exhibited a complete response to a cancer treatment may still have cancer, as evidenced by a subsequent relapse.

“Systemic delivery,” as used herein, refers to delivery that leads to a broad biodistribution of a compound within an organism. Systemic delivery means that a useful, preferably therapeutic, amount of a compound is exposed to most parts of the body. To obtain broad biodistribution generally requires a route of introduction such that the compound is not rapidly degraded or cleared (such as by a first pass organ (e.g., liver, lung, etc.) or by rapid, nonspecific cell binding) before reaching a disease site. Systemic delivery of liposome-encapsulated chemotherapeutic alkaloids is preferably obtained by intravenous delivery.

A “stable disease” is a state wherein a therapy causes cessation of growth or prevalence of a tumor or tumors as measured by the usual clinical, radiological and biochemical means, although there is no regression or decrease in the size or prevalence of the tumor or tumors, i.e., cancer that is not decreasing or increasing in extent or severity.

“Maintenance therapy” refers to an extended therapy that is typically administered after a primary treatment for a disease, and which is administered in order to deter the recurrence or worsening of the disease.

“Partial response” or “partial remission” refers to the amelioration of a cancerous state, as measured by tumor size and/or cancer marker levels, in response to a treatment. Typically, a “partial response” means that a tumor or tumor-indicating blood marker has decreased in size or level by about 50% in response to a treatment. The treatment can be any treatment directed against cancer, but typically includes chemotherapy, radiation therapy, hormone therapy, surgery, cell or bone marrow transplantation, immunotherapy, and others. The size of a tumor can be detected by clinical or by radiological means. Tumor-indicating markers can be detected by means well known to those of skill, e.g., ELISA or other antibody-based tests.

A “complete response” or “complete remission” means that a cancerous state, as measured by, for example, tumor size and/or cancer marker levels, has disappeared following a treatment such as chemotherapy, radiation therapy, hormone therapy, surgery, cell or bone marrow transplantation, or immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

“Chemotherapy” refers to the administration of chemical agents that inhibit the growth, proliferation and/or survival of cancer cells. Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapy can include any chemical agent that inhibits, or is designed to inhibit, a cancerous cell or a cell likely to become cancerous. Such agents are often administered, and are often most effective, in combination, e.g., in the formulation CHOP.

“Radiation therapy” refers to the administration of radioactivity to an animal with cancer. Radiation kills or inhibits the growth of dividing cells, such as cancer cells.

“Surgery” is the direct removal or ablation of cells, e.g. cancer cells, from an animal. Most often, the cancer cells are in the form of a tumor, which is removed from the animal.

“Hormone therapy” refers to the administration of compounds that counteract or inhibit hormones, such as estrogen or androgen, that have a mitogenic effect on cells. Often, these hormones act to increase the cancerous properties of cancer cells in vivo.

“Immunotherapy” refers to methods of enhancing the ability of an animal's immune system to destroy cancer cells within the animal.

A “free-form” therapeutic agent, or “free” therapeutic agent, refers to a therapeutic agent that is not liposome-encapsulated. Usually, a drug is presumed to be “free, or in a “free-form,” unless specified otherwise. A vinca alkaloid in free form may still be present in combination with other reagents, however, such as other chemotherapeutic compounds, a pharmaceutical carrier, or complexing agents, i.e. as used herein the term only specifically excludes lipid formulations of the vinca alkaloids.

### III. Diseases and Conditions Treatable with Lipid-Encapsulated Chemotherapeutic Agents (e.g., Chemotherapeutic Alkaloids)

The methods described herein can be used to treat or prevent any disease or condition associated with angiogenesis. In a preferred embodiment, the disease or condition is cancer. Any type of cancer can be treated using these methods including, but not limited to, lung cancer, breast cancer, gastrointestinal cancers, prostate cancer, liver cancer, colorectal cancer, lymphomas, leukemias, skin cancer, myelomas, kidney cancer, neuroblastomas, small cell lung cancer, bladder cancer, bone cancer, CNS cancers, ovarian cancer, pancreatic cancer, sarcomas, testicular cancer, or any other type of cancer.

The present methods can be used as a first-line treatment for the cancer, or can be used as a maintenance therapy, i.e., the methods can be applied subsequent to a first line treatment in order to prevent the progression, reappearance, continued presence, or metastasis of a tumor. In certain embodiments of the present invention, liposome encapsulated chemotherapeutic agents, such as alkaloids, are employed against “resistant” cancers, i.e., cancers which have previously exhibited a complete response to a treatment, but which subsequently manifest a resistance to a second or later course of treatment. Because the present methods are directed to the prevention of angiogenesis, rather than to the eradication of the tumor itself, even tumors that are resistant to the chemotherapeutic agent used in the present methods can be targeted.

The present methods can also be used to treat or prevent other diseases or conditions associated with angiogenesis. For example, pathological angiogenesis, induced by local ischemia, can occur in ocular diseases (diabetic retinopathy, retinopathy of premature infants, age-related macular degeneration); vascular diseases (ischemic heart disease and atherosclerosis); and chronic inflammatory disorders (psoriasis and rheumatoid arthritis). Other diseases and conditions include rubeotic glaucoma, interstitial keratitis, corneal graft failure, restenosis, and vasculopathies including hemangiomas and systemic vasculitis. Any of these conditions and diseases can be treated using the herein-provided methods.

### IV. Vinca Alkaloids, Other Alkaloids and Other Agents

The present invention can include the use of any chemotherapeutic agent. In a presently preferred embodiment, the present invention includes the use of a chemotherapeutic alkaloid. The present invention can include the use of any naturally occurring alkaloid, including vinca alkaloids, or any synthetic derivative of a naturally occurring alkaloid. Vinca alkaloids include, but are not limited to, vinblastine, vincristine, vindoline, vindesine, vinleurosine, vinrosidine, vinorelbine, or derivatives thereof (see, e.g., the Merck Index, 11^{th} Edition (1989) entries 9887, 9891, and 9893, for vinblastine, vincristine, and vindoline). Examples of other suitable chemotherapeutic agents include, but are not limited to, the podophyllins, podophyllotoxins, and derivatives thereof (e.g., etoposide, etoposide phosphate, teniposide, etc.), the camptothecins (e.g., irinotecan, topotecan, etc.) the taxanes (taxol, etc.), and derivatives thereof. All of the above compounds are well known to those of skill and are readily available from commercial sources, by synthesis, or by purification from natural sources.

In preferred embodiments, the vinca alkaloid used in the present invention is vincristine. Vincristine, also known as leurocristine sulfate, 22-oxovincaleukoblastine, Kyocristine, vincosid, vincrex, oncovin, Vincasar PFS®, or VCR, is commercially available from any of a number of sources, e.g., Pharmacia & Upjohn, Lilly, IGT, etc. It is often supplied as vincristine sulfate, e.g., as a 1 mg/mL solution.

The present invention can comprise the use of a single chemotherapeutic agent (e.g., alkaloid) or multiple, co-administered agents (e.g., alkaloids). In a preferred embodiment, one or more vinca alkaloids can be combined with other compounds or molecules, such as other anti-neoplastic agents. In certain embodiments, such combinations of vinca alkaloids and/or other compounds can be made prior to liposomal formulation, thereby creating a combination within a single liposome. In other embodiments, liposome-encapsulated vinca alkaloids are formulated and subsequently combined with the other molecules, which can themselves be free-form or liposome-encapsulated.

The selection of a particular chemotherapeutic agent (e.g., chemotherapeutic alkaloid) is made based on a number of competing considerations, including, but not limited to, the following:

i) the agent must modulate angiogenesis at the administered dose (i.e., inhibit or reduce angiogenesis or, alternatively, destructively accelerate angiogenesis);

ii) the administered dose of the agent, e.g., the alkaloid, (e.g., a low dose) must have low collateral toxicity such that it may be administered as a chronic maintenance therapy for the desired period of time before the patient suffers a dose limiting toxic response (i.e., long term chronic use is possible before the maximum total cap on dosing, if any, is reached);

iii) the dose of the agent, e.g., the alkaloid, is preferably administered as rarely as possible for the convenience of the patient (i.e., long circulating forms of the agent with extended payload delivery are preferred); and

iv) the dose of the agent, e.g., the alkaloid, is preferably administered so that the highest concentrations of the agent accumulate at the neovasculature of the disease site (i.e., for blood borne diseases, intravenous administration is preferred over oral); also, formulations which preferentially accumulate at disease sites are preferred.

### V. Lipids

Any of a number of lipids can be used to prepare the liposomes of the present invention, including amphipathic, neutral, cationic, and anionic lipids. Such lipids can be used alone or in combination, and can also include bilayer stabilizing components such as polyamide oligomers (see, e.g., U.S. patent application Ser. No. 09/218,988, filed Dec. 22, 1998 (now U.S. Pat. No. 6,320,017), entitled “Polyamide Oligomers,” by Steven Ansell, the teachings of which are incorporated herein by reference), peptides, proteins, detergents, lipid-derivatives, such as PEG coupled to phosphatidylethanolamine and PEG conjugated to ceramides (see, U.S. application Ser. No. 08/485,608, the teachings of which are incorporated herein by reference). In a preferred embodiment, cloaking agents, which reduce elimination of liposomes by the host immune system, can also be included, such as polyamide-oligomer conjugates, e.g., ATTA-lipids, (see, U.S. patent application Ser. No. 08/996,783, filed Feb. 2, 1998, the teachings of which are incorporated herein by reference) and PEG-lipid conjugates (see, U.S. patent application Ser. Nos. 08/486,214, 08/316,407 and 08/485,608, the teachings of which are incorporated herein by reference).

Any of a number of neutral lipids can be included, referring to any of a number of lipid species which exist either in an uncharged or neutral zwitterionic form at physiological pH, including diacylphosphatidylcholine, diacylphosphatidylethanolamine, ceramide, sphingomyelin, cephalin, cholesterol, cerebrosides, and diacylglycerols.

In preferred embodiments, the lipid used is sphingomyelin. In particularly preferred embodiments, the lipid comprises sphingomyelin and cholesterol. In such embodiments, the ratio of sphingomyelin to cholesterol is typically between about 75/25 (mol % sphingomyelin/mol % cholesterol) and about 50/50 (mol % sphingomyelin/mol % cholesterol), preferably between about 70/30 and 55/45 (mol % sphingomyelin/mol % cholesterol), and most preferably about 55/45 (mol % sphingomyelin/mol % cholesterol). Such ratios, may be altered, however, by the addition of other lipids into the present formulations.

Cationic lipids, which carry a net positive charge at physiological pH, can readily be incorporated into liposomes for use in the present invention. Such cationic lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3β-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”); and N-(1,2-dimyristyloxyprop-3-yl)-N,N-dimethyl-N-hydroxyethyl ammonium bromide (“DMRIE”). Additionally, a number of commercial preparations of cationic lipids can be used, such as LIPOFECTIN (including DOTMA and DOPE, available from GIBCO/BRL), LIPOFECTAMINE (comprising DOSPA and DOPE, available from GIBCO/BRL), and TRANSFECTAM (comprising DOGS, in ethanol, from Promega Corp.).

Anionic lipids suitable for use in the present invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In numerous embodiments, amphipathic lipids will be used. “Amphipathic lipids” refer to any suitable material, wherein the hydrophobic portion of the lipid material orients into a hydrophobic phase, while the hydrophilic portion orients toward the aqueous phase. Such lipids include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

The liposomes used in the present invention can be multilamellar or unilamellar, which can be formed using the methods disclosed herein and other methods known to those of skill in the art.

Also suitable for inclusion in the present invention are programmable fusion lipid formulations. Such formulations have little tendency to fuse with cell membranes and deliver their payload until a given signal event occurs. This allows the lipid formulation to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the liposome membrane over time. By the time the formulation is suitably distributed in the body, it has lost sufficient cloaking agent so as to be fusogenic. With other signal events, its is desirable to choose a signal that is associated with the disease site or target cell, such as increased temperature at a site of inflammation.

### VI. Making Liposomes

A variety of methods are available for preparing liposomes as described in, e.g., Szoka, et al., Ann. Rev. Biophys. Bioeng., 9:467 (1980), U.S. Pat. Nos. 4,186,183, 4,217,344, 4,235,871, 4,261,975, 4,485,054, 4,501,728, 4,774,085, 4,837,028, 4,946,787, PCT Publication No. WO 91/17424, Deamer, et al., Biochim. Biophys. Acta, 443:629-634 (1976); Fraley, et al., Proc. Natl. Acad. Sci. USA, 76:3348-3352 (1979); Hope, et al., Biochim. Biophys. Acta, 812:55-65 (1985); Mayer, et al., Biochim. Biophys. Acta, 858:161-168 (1986); Williams, et al., Proc. Natl. Acad. Sci., 85:242-246 (1988), the text Liposomes, Marc J. Ostro, ed., Marcel Dekker, Inc., New York, 1983, Chapter 1, and Hope, et al., Chem. Phys. Lip., 40:89 (1986), all of which are incorporated herein by reference. Suitable methods include, but are not limited to, sonication, extrusion, high pressure/homogenization, microfluidization, detergent dialysis, calcium-induced fusion of small liposome vesicles, and ether-infusion methods, all of which are well known in the art.

One method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents, such as deoxycholate.

Unilamellar vesicles can be prepared by sonication or extrusion. Sonication is generally performed with a tip sonifier, such as a Branson tip sonifier, in an ice bath. Typically, the suspension is subjected to severed sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass. Unilamellar vesicles can also be made by dissolving phospholipids in ethanol and then injecting the lipids into a buffer, causing the lipids to spontaneously form unilamellar vesicles. Also, phospholipids can be solubilized into a detergent, e.g., cholates, Triton X, or n-alkylglucosides. Following the addition of the drug to the solubilized lipid-detergent micelles, the detergent is removed by any of a number of possible methods including dialysis, gel filtration, affinity chromatography, centrifugation, and ultrafiltration.

Following liposome preparation, the liposomes which have not been sized during formation may be sized to achieve a desired size range and relatively narrow distribution of liposome sizes. A size range of about 0.2-0.4 microns allows the liposome suspension to be sterilized by filtration through a conventional filter. The filter sterilization method can be carried out on a high through-put basis if the liposomes have been sized down to about 0.2-0.4 microns.

Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method that relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve gradual reduction in liposome size. For use in the present invention, liposomes having a size ranging from about 0.05 microns to about 0.40 microns are preferred. In particularly preferred embodiments, liposomes are between about 0.05 and about 0.2 microns.

In preferred embodiments, empty liposomes are prepared using conventional methods known to those of skill in the art.

Typically, as discussed infra, the liposomes used in the present invention will comprise a transmembrane potential, whereby the chemotherapeutic alkaloids are effectively loaded into and retained by the liposome. In preferred embodiments, the potential will be effected by creating a pH gradient across the membrane. In particularly preferred embodiments, the pH is lower at the interior of the liposomes than at the exterior. Such gradients can be achieved, e.g., by formulating the liposomes in the presence of a buffer with a low pH, e.g., having a pH between about 2 and about 6, and subsequently transferring the liposomes to a higher pH solution. In preferred embodiments, the pH is between about 3 and 5, and in most preferred embodiments, the pH is about 4. Any of a number of buffers can be used, such as citrate.

Subsequently, before or after sizing, the external pH can be raised, e.g., to about 7 or 7.5, by the addition of a suitable buffer, such as a sodium phosphate buffer. Raising the external pH creates a pH gradient across the liposomal membrane, thereby promoting efficient drug loading and retention.

Liposomes prepared according to these methods can be stored for substantial periods of time prior to drug loading and administration to a patient. For example, liposomes can be dehydrated, stored, and subsequently rehydrated, loaded with one or more vinca alkaloids, and administered. Dehydration can be accomplished, e.g., using standard freeze-drying apparatus, i.e., they are dehydrated under low pressure conditions. Also, the liposomes can be frozen, e.g., in liquid nitrogen, prior to dehydration. Sugars can be added to the liposomal environment, e.g., to the buffer containing the liposomes, prior to dehydration, thereby promoting the integrity of the liposome during dehydration. See, e.g., U.S. Pat. No. 5,077,056 or 5,736,155.

In numerous embodiments, the empty liposomes are first formulated in low pH buffer, and then manipulated in one of a variety of ways to obtain liposomes of the desired size. Methods for sizing liposomes include sonication, by bath or by probe, or homogenization. Preferably, following such treatments, the liposomes are between about 0.05 to 0.45 microns. Most preferably, the liposomes are between about 0.05 and about 0.2 microns. Such sized liposomes can then be sterilized by filtration. Also, particle size distribution can be monitored by conventional laser-beam particle size discrimination or the like. In addition, methods of reducing liposome sizes to a relatively well defined size distribution are known, e.g., one or more cycles of extrusion of the liposomes through a small-pore polycarbonate membrane or an asymmetric ceramic membrane.

### VII. Preparation of Liposome-Encapsulated Agents

Any of a number of methods can be used to load the chemotherapeutic agents, such as alkaloids and/or other drugs, into the liposomes. Such methods include, e.g., an encapsulation technique and a transmembrane potential loading method. Generally, following such methods, the chemotherapeutic agents, such as vinca alkaloids, are present at about 0.05 mg/mL to about 0.1 mg/mL. Preferably, the agents, e.g., the alkaloids, are present at about 0.1 to 0.5 mg/mL and, more preferably, 0.15 to 0.2 mg/mL.

In one encapsulation technique, the drug and liposome components are dissolved in an organic solvent in which all species are miscible and concentrated to a dry film. A buffer is then added to the dried film and liposomes are formed having the drug incorporated into the vesicle walls. Alternatively, the drug can be placed into a buffer and added to a dried film of only lipid components. In this manner, the drug will become encapsulated in the aqueous interior of the liposome. The buffer which is used in the formation of the liposomes can be any biologically compatible buffer solution of, for example, isotonic saline, phosphate buffered saline, or other low ionic strength buffers. The resulting liposomes encompassing the chemotherapeutic agents, e.g., the vinca alkaloids, can then be sized as described above.

Transmembrane potential loading has been described in detail in U.S. Pat. Nos. 4,885,172, 5,059,421, 5,171,578, and 5,837,282 (which teaches ionophore loading), the teachings of each of which is incorporated herein by reference. Briefly, the transmembrane potential loading method can be used with essentially any conventional drug which can exist in a charged state when dissolved in an appropriate aqueous medium. Preferably, the drug will be relatively lipophilic so that it will partition into the liposome membranes. A transmembrane potential is created across the bilayers of the liposomes or protein-liposome complexes and the drug is loaded into the liposome by means of the transmembrane potential. The transmembrane potential is generated by creating a concentration gradient for one or more charged species (e.g., Na⁺, K⁺, and/or H⁺) across the membranes. This concentration gradient is generated by producing liposomes having different internal and external media and has an associated proton gradient. Drug accumulation can then occur in a manner predicted by the Henderson-Hasselbach equation.

Preferred methods of preparing liposome-encapsulated vinca alkaloids for use in the present invention are discussed, e.g., in U.S. Pat. Nos. 5,741,516, 5,814,335 and 5,543,152, each of which is assigned to Inex Pharmaceuticals Corp. and is incorporated herein by reference. In a preferred embodiment, liposomal vinca alkaloids are prepared prior to use from a kit including 3 or more vials. At least one of the vials contains a vincristine solution containing, e.g., 1 mg/mL, 2 mg/mL, or 5 mg/mL, preferably 1 mg/mL, vincristine sulfate in buffer containing, e.g., 100 or 200 mg/mL mannitol (obtainable from, e.g., SP Pharmaceuticals LLC, Albuquerque, N.M.; other excipients that are pharmaceutically acceptable, and in which vincristine remains stable for extended periods, can also be used) and sodium acetate adjusted to pH 3.5 to 5.5, or preferably pH 4.5 to pH 4.7. One of the vials contains a solution containing liposomes comprising sphingomyelin and cholesterol (each of which is commercially available, e.g., from NEN Life Sciences, Avanti Polar Lipids, etc.) and suspended in a 300 mM citrate buffer at, e.g., pH 4.0. Another vial or vials contains a alkaline phosphate buffer (e.g., pH 9.0) such as dibasic sodium phosphate, 14.2 mg/ml (20 ml/vial).

In other preferred embodiments, a kit is used that contains 2 vials containing components that can be used to formulate the claimed liposome-encapsulated chemotherapeutic agents, e.g., alkaloids, or a kit containing 1 vial containing a stable preparation of liposomes comprising pre-loaded agents, e.g., alkaloids. Such stable preparations can be accomplished in any of a number of ways, including, but not limited to, (1) a hydrated preparation stored at ambient temperatures or refrigerated and which contains one or more modifications or components to enhance chemical stability, e.g., antioxidants; (2) a hydrated preparation that was frozen and which includes a suitable excipient to protect from freeze/thaw-induced damage; or (3) a lyophilized preparation. Typically, any of the above-described kits also contain instructions for use as well as clean-up disposal materials.

To prepare the liposomes, for instance, the vincristine sulfate and liposome solutions are each added to a sterile vial and mixed, at an appropriate concentration ratio, e.g., 0.01/1.0 to 0.2/1.0 (wt. vinca alkaloid/wt. lipid) and, more preferably, 0.05/1.0 to 0.1/1.0 (wt. vinca alkaloid/wt. lipid). (It will be readily appreciated by those of skill in the art that the foregoing exemplar ratios are applicable to other chemotherapeutic agents as well). The mixture is mixed, e.g., by inverting the vial multiple times. Following the formation of the liposomes in low pH buffer, and either before or after the sizing of the liposomes, the liposomes are introduced into buffer of a higher pH, e.g., a sodium phosphate buffer, thereby creating a pH gradient across the liposome surface. In preferred embodiments, the external environment of the liposomes is between about pH 7.0 and about pH 7.5. The liposomes and vinca alkaloids can be mixed for an amount of time sufficient to achieve the desired alkaloid/lipid ratio. The mixture can be mixed, e.g., by multiple inversions, and heated to temperatures between about 55° C. and about 80° C., preferably between about 60° C. and about 65° C., for about 5, 10, or more minutes. Such treatment causes greater than about 90% of the vincristine to become entrapped within the liposome.

In other embodiments, these steps are followed at a larger scale, and loaded liposomal vincristine is supplied to, e.g., a hospital pharmacy in ready-to-administer format. Such larger scale formulations may be prepared from different starting materials than those described for the kit; in particular, the buffers may be different.

### VIII. Targeting Liposomes

In certain embodiments, it is desirable to target the liposomes of this invention using targeting moieties that are specific to a cell type or tissue. Targeting of liposomes using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044, the teachings of which are incorporated herein by reference). The targeting moieties can comprise the entire protein or fragments thereof.

Targeting mechanisms generally require that the targeting agents be positioned on the surface of the liposome in such a manner that the target moiety is available for interaction with the target, for example, a cell surface receptor. The liposome is designed to incorporate a connector portion into the membrane at the time of liposome formation. The connector portion must have a lipophilic portion that is firmly embedded and anchored into the membrane. It must also have a hydrophilic portion that is chemically available on the aqueous surface of the liposome. The hydrophilic portion is selected so as to be chemically suitable with the targeting agent, such that the portion and agent form a stable chemical bond. Therefore, the connector portion usually extends out from the liposomal surface and is configured to correctly position the targeting agent. In some cases, it is possible to attach the target agent directly to the connector portion, but in many instances, it is more suitable to use a third molecule to act as a “molecular bridge.” The bridge links the connector portion and the target agent off of the surface of the liposome, thereby making the target agent freely available for interaction with the cellular target.

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990)). Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

### IX. Administration of Lipid-Encapsulated Agents (e.g., Alkaloids)

Liposome-encapsulated agents (e.g., alkaloids) can be administered in any of a number of ways, including parenteral, intravenous, systemic, local, intratumoral, intramuscular, subcutaneous, intraperitoneal, inhalation, or any such method of delivery. In preferred embodiments, the pharmaceutical compositions are administered intravenously by injection. In one embodiment, a patient is given an intravenous infusion of the liposome-encapsulated agent, e.g., alkaloid, (single agent) through a running intravenous line over, e.g., 30 minutes, 60 minutes, 90 minutes, or longer. In preferred embodiments, a 60 minute infusion is used. Such infusions can be given periodically, e.g., once every 1, 3, 5, 7, 10, 14, 21, or 28 days or longer, preferably about once every 7 days. As used herein, each administration of a liposomal chemotherapeutic agent (e.g., alkaloid) is considered one “course” of treatment.

Suitable formulations for use in the present invention can be found, e.g., in Remington's Pharmaceutical Sciences, Mack Publishing Company, Philadelphia, Pa., 17^{th} Ed. (1985). Often, intravenous compositions will comprise a solution of the liposomes suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. These compositions can be sterilized by conventional sterilization techniques, such as filtration. The compositions may contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, triethanolamine oleate, etc. These compositions can be sterilized using the techniques referred to above, or can be produced under sterile conditions. The concentration of liposomes in the carrier can vary. Generally, the concentration will be about 20-200 mg/mL, however persons of skill can vary the concentration to optimize treatment with different liposome components or for particular patients. For example, the concentration may be increased to lower the fluid load associated with treatment.

### X. Determining an Optimal Course of Treatment

The amount of chemotherapeutic agents, e.g., alkaloids, administered per dose, and the frequency of administration, is typically selected based on an empirical determination that is well within the capability of one of skill in the art. Generally, because the present methods involve the administration of the compounds at an increased frequency compared to traditional regimens, the amount of the compound given at any one time will be typically lower than according to conventional protocols. For example, conventional regimens often involve the administration of a maximum tolerable dose of a chemotherapeutic agent once or several times over a short period, followed by a relatively long “rest” period in which the body is allowed to recover, and during which the compound is cleared from the body. In contrast, the present methods often involve a more sustainable dosage form that can be administered at relatively high frequency over an indefinite amount of time, and, as such, must be administered at a dosage form that, even over long periods of administration, are preferably non-toxic or only minimally toxic. Accordingly, the dosage forms used in the present invention are typically lower than those used in conventional therapies.

A suitable starting point for determining an optimal regimen is to calculate the appropriate dosage for an increased frequency based upon the conventional dosage. For example, if a conventional therapy calls for administration of 30 mg/m² every three weeks, which has been previously determined to be the maximum dosage possible, then one may begin with administration of 10 mg/m² every week, or 5 mg/m² twice per week, so that the overall amount of administered drug is the same. With this as a starting point, either the frequency and/or individual dosage form can be altered to identify a frequency/dosage combination that allows maximum efficacy, convenience, and minimum toxicity. In some cases, the overall dosage will be greater than for conventional therapies. Another suitable method for determining an initial dose is to use a given fraction of the MTD for the alkaloid, depending on the intended frequency of administration, the convention protocol, and other factors. For example, a starting dosage of 0.001, 0.005, 0.01, 0.02, 0.05, 0.1, 0.2, or more of the MTD can be used. Determining, monitoring, and, if necessary, altering a suitable regimen can readily be accomplished by one of skill in the art.

The choice of amount and frequency per dose will also depend on a number of additional factors, such as the medical history of the patient, the use of other therapies, and the nature of the disease. In certain embodiments, an initially low dose will be given, which can be increased based on the response and/or tolerance of the patient to the initial dose. For example, for liposomal vincristine, <0.05, 0.05, 0.1, 0.2, 0.3, 0.4, 0.5, 1.0 mg/m², 1.5 mg/m2 (i.e., mg vincristine, per m² body surface area) or more can be administered. In preferred embodiments, patients are administered a dose of from about 0.1 to about 0.2 mg/m2 (0.2 mg/m2 vincristine is about the same as 0.01 mg/kg for an average 70 kg patient; 0.01 mg/kg vincristine is about the same as 0.2 mg/kg lipid dose for a d/l ratio of about 0.05). (It will be readily appreciated by those of skill in the art that the foregoing exemplar doses for vincristine are applicable to the other chemotherapeutic agents disclosed herein. In many instances, such dose ranges can be used as a starting point for optimizing the actual dose used.) In addition, the frequency and dosage of administration can be influenced by questions of convenience. For example, when the formulations are administered by injection, i.e., in an outpatient setting, a less frequent administration (e.g., at most about once per week) is preferred. When the formulations are administered by the patient alone, e.g., for an oral formulation, more frequent administrations (e.g., twice weekly, daily, etc.) may be preferred).

Patients typically will receive at least 4, 6, 8, 10, 15, 20, 40, 80, or more courses of such treatment, depending on the response of the patient to the treatment. In single agent regimens, total courses of treatment are determined by the patient and physician based on observed responses and toxicity. Greater numbers may be warranted in certain cases.

Because vincristine and other chemotherapeutic alkaloid dosages are often limited by neurotoxicity in humans, it is sometimes useful to co-administer liposomal alkaloids, e.g., vincristine, with a treatment for neurotoxicity. This treatment may be prophylactic or therapeutic. An example is the administration of Neurontin™ gabapentin (Parke-Davis), or neurotonin, for treatment of neuropathic pain, e.g., 100-200 mg Neurontin™ is administered 3 times per day to an adult patient. If neuropathic pain improves, then liposomal vincristine treatments may continue. Because this type of prophylactic or therapeutic treatment is intended only to treat side-effects of liposomal vincristine, it is considered separately from the combination therapies set forth below.

It is important to emphasize that the encapsulation of the chemotherapeutic alkaloids in liposomes imparts a dramatic benefit on the administration of the alkaloids for the herein-described diseases and conditions. For example, the enhanced stability and prolonged delivery of the alkaloids in vivo following liposomal encapsulation leads to an increased persistence of the compound in the body, which is important for an anti-angiogenic effect. In addition, liposomal encapsulation provides decreased toxicity of the alkaloids, thereby allowing the administration of a greater amount of alkaloid per dose and over time. Further, liposomal formulations can provide improved biodistribution of the alkaloid, e.g., increased intra-tumoral concentrations, thereby providing more efficacious administration of the compounds.

### XI. Animal Models

Any therapeutic regimen or strategy that falls within the scope of this invention can be readily assessed using any of a number of different experimental animal models. For example, to evaluate the ability of a particular regimen to treat or prevent cancer, any of a large number of animal models for cancer can be used. Often, cancerous cells (e.g., cell lines derived from a tumor) or any cells that are capable of forming a tumor in vivo, are introduced into an animal, e.g., a mouse. The effect of a compound on tumorigenesis is then assessed by, e.g., allowing tumors to grow and then administering the compound to determine whether the growth of the tumors is slowed, arrested, or reversed. By introducing cells of varying types (e.g., breast cancer cells, lung cancer cells, lymphoma cells, etc.), and by varying the method and site of introduction of the cells (e.g., intravenous, subcutaneous, etc.) the effect of the compound on various tumor types can be assessed. See, e.g., Examples I and II, infra.

In one embodiment, the effect of a particular administration protocol on angiogenesis can, e.g., be directly assessed by introducing tumorigenic cells that are resistant to a particular compound, e.g., either naturally or by selection in vitro or in vivo. When such cells are introduced into an animal, and the compound is administered using the present methods, any effect on the growth of the tumor necessarily occurs by an inhibition of angiogenesis, rather than by an effect on the tumor cells themselves. Examples of cells that can be used in such methods include, but are not limited to, Lewis Lung carcinoma cells and NCI-H69 small cell lung cancer cells, which can, e.g., be selected in vitro or in vivo for resistance to the alkaloid.

Another type of model involves the use of cancer-causing mutations in animals. For example, mutations removing tumor suppressor genes, or expressing oncogenes, often lead to cancer in experimental animals. The ability of a compound or formulation to prevent or treat the cancer can similarly be assessed using such animals. All of these methods are well known to those of skill in the art.

### XII. Combination Therapies

In numerous embodiments, liposome-encapsulated chemotherapeutic alkaloids are administered in combination with one or more additional compounds or therapies. For example, multiple vinca alkaloids can be co-administered, or one or more vinca alkaloids can be administered in conjunction with another therapeutic compound, such as cyclophosphamide, doxorubicin, prednisone, other chemotherapeutic agents such as the taxanes, camptothecins, and/or podophyllins, other therapeutic agents such as antisense drugs or anti-tumor vaccines. In a preferred embodiment, liposome-encapsulated vincristine is co-administered with cyclophosphamide, doxorubicin, and prednisone. In certain embodiments, multiple compounds are loaded into the same liposomes. In other embodiments, liposome-encapsulated vinca alkaloids are formed individually and subsequently combined with other compounds for a single co-administration. Alternatively, certain therapies are administered sequentially in a predetermined order, such as in CHOP or lipo-CHOP (i.e., CHOP comprising liposomal vincristine). Liposome-encapsulated vincristine can also be formulated in a CVP combination, or cyclophosphamide-vincristine-prednisone.

Liposome-encapsulated vinca alkaloids can also be combined with other anti-tumor agents such as monoclonal antibodies including, but not limited to, Oncolym™ (Techniclone Corp. Tustin, Calif.) or Rituxan™ (IDEC Pharmaceuticals), Bexxar™ (Coulter Pharmaceuticals, Palo Alto, Calif.), or IDEC-Y2B8 (IDEC Pharmaceuticals Corporation). In addition, liposome-encapsulated vinca alkaloids can be administered along with one or more non-molecular treatments such as radiation therapy, bone marrow transplantation, hormone therapy, surgery, etc.

In a preferred embodiment, liposome encapsulated vinca alkaloids are administered in combination with an anti-cancer compound or therapy which provides an increased or synergistic improvement in tumor reduction based on mechanism of action and non-overlapping toxicity profiles. In particular, liposomal vinca alkaloids can be delivered with a taxane, which optionally may also be a liposomal taxane. While it is thought that vinca alkaloids depolymerize microtubules and taxanes stabilize microtubules, the two compounds have been found to act synergistically in the impairment of tumor growth, presumably because both are involved in the inhibition of microtubule dynamics. See, Dumontet, et al. J. Clin. Onc. 17:1061-1070 (1999). Liposomal formulations of the alkaloids according to the present invention will thus significantly diminish the myeloid and neurologic toxicity associated with the sequential administration of free form vinca alkaloids and taxanes.

In another preferred embodiment, the liposome-encapsulated chemotherapeutic alkaloids are co-administered with another angiogenesis inhibitor. Any such inhibitor can be used, including, but not limited to, thrombospondin, internal fragments of thrombospondin, angiostatin, endostatin, vasostatin, vascular endothelial growth factor inhibitor (VEGI), fragment of platelet factor 4 (PP4), derivative of prolactin, restin, proliferin-related protein (PRP), SPARC cleavage product, osteopontin cleavage product, interferon α, interferon β, meth 1, meth I, angiopoietin-2, anti-thrombin III fragment, COL-3, squalamine, combretastatin, PTK787/ZK2284, CAI, PIK787/2K22584, CGS-27023A, TNP-470, thalidomide, SU5416, vitaxin, IL-12, EMD121974, marimastat, AG3340, neovastat/AE941, anti-VEGF Ab, and IM862. See, e.g., Griffioen, et al., Pharmacol. Rev., 52:237-68 (2000); Rosen, Oncologist, 5 Suppl. 1:20-7 (2000).

Other combination therapies known to those of skill in the art can be used in conjunction with the methods of the present invention.

### XIII. EXAMPLES

### A. Example I

### Administration of Liposome-encapsulated Alkaloids to an Animal Model of Orthotopic Metastasis.

The murine CT26 orthotopic tumor model is well characterized, is employed extensively in the evaluation of therapeutic oncology agents, and reflects late stage disease progression of colorectal cancer patients who develop metastatic lesions in the liver following resection of the primary tumor. In this experimental metastatic tumor model, CT26 tumor cells are implanted intrasplenically, resulting in seeding of tumor cells directly to the liver. Histological examination of the liver demonstrates metastatic growth from which the animals eventually succumb. Overall tumor burden of the diseased animals correlates with the survival endpoint, i.e., duration of survival is directly related to growth of CT26 hepatic lesions.

Using the CT26 model, the evaluation of a particular regimen is typically performed as follows. Following implantation and establishment of tumor cells in the liver, the liposomal alkaloid is administered intravenously, and the dose and frequency of administration is determined empirically, but is optimized according to evaluation of the therapeutic effect. In addition, the magnitude of the dose constitutes a balance with the toxicity observed upon repeat administration.

Metastatic lesions require angiogenesis in order to expand beyond approximately 1-2 mm³. The continued exposure of metastatic lesions to any of the herein-described formulations inhibits endothelial cell growth (angiogenesis) and prevents tumor growth. Alternatively, hepatic lesions are allowed to develop more extensively, with expected concomitant growth of new vasculature. Administration of the formulation at this stage, and the demonstration of inhibition of tumor growth, would establish the capacity of the formulation to inhibit the growth of established experimental metastases.

The anti-tumor activity of the liposome-encapsulated alkaloid is assessed by monitoring duration of survival of tumor-bearing animals relative to negative controls (animals receiving saline or “empty” liposomes, i.e., devoid of alkaloid). Animals receiving repeated administration of the formulated alkaloid survive longer than control treated animals. Evaluation of total tumor burden (average liver weights in experimental animals compared to controls) is used to confirm the correlation of tumor growth with survival. The actual number of metastatic lesions may not be different between the experimental animals and controls, however, as the present methods are expected to halt the growth of micrometastatic lesions, but not necessarily cause tumor eradication.

### B. Example II

### Subcutaneous, Ectopic Implantation Models

Numerous tumor cell lines both of murine or human origin grow to form established tumors replete with tumor vasculature following implantation of cells in the subcutaneous compartment. In addition, tumors of various cancer types can be used in this model system. Typically, cells propagated in vitro are implanted subcutaneously in the hind flank of the animal, and tumor volumes are measured over time until the tumor size reaches a toxic level, at which time the animal must be euthanized.

Following subcutaneous implantation, tumors are allowed to expand to a measurable size (typically 100 mm³), at which time the liposome-encapsulated chemotherapeutic alkaloid is administered intravenously, and the dose and frequency of administration are determined empirically, but are optimized according to evaluation of the therapeutic effect as well as toxicity of repeated dosing. Tumor growth is monitored over time and the data expressed as percent tumor growth inhibition or tumor growth delay. Using this experimental system, low dose maintenance therapy is evaluated according to the ability to inhibit or stabilize tumor growth. These models reflect the capacity of the formulation to stabilize the growth of well established tumors with extensive tumor vasculature.

It is expected that the present formulation reduce or stabilize tumor growth when compared to negative controls.

### C. Example III

### Efficacy of VSLI in Treating LX-1 Tumors in a Multiple High Dose Treatment Schedule

This example illustrates a protocol that can be used to evaluate the efficacy of VSLI in treating LX-1 tumors in a multiple high dose treatment schedule.

LX-1 cells were obtained from the NCI and serially passaged in mice for 5 cycles before being used in this experiment. Tumors from passage animals were harvested when they reach 10-15 mm in diameter (300-600 mm³). The tumor was resected and cut in pieces of 2-3 mm³ (0.014-0.17 g). Tumor fragments of the appropriate size were implanted into the 60 mice (60 NCR nu/nu mice, female, 20-23 g, 5-6 weeks old) using a 10G trokar while mice were anesthetized with halothane. Sixty mice were implanted with tumor, but up to 20% were excluded at time of treatment due to small tumor size.

When the average tumor size reached 100 mm³, the mice were randomized into groups (4 mice per group). Mice with very small tumors were excluded from the experiment. Twenty percent of mice implanted were excluded due to small tumor size, therefore, only 48 of the 60 mice implanted were treated. Mice were given three treatments of VSLI or vincristine at the appropriate dose (based on their body weight) every three days or every five days. Injections were given intravenously in the tail vein in approximately 200 μl.

When tumors reached an average size of 100 mm³ (11 days after implantation) mice were given their first treatment with either VSLI or vincristine. Subsequent treatments were given following the schedules listed below. Mice were weighed on the day of injection and the average weight of the mice in each group was used to calculate the concentration of VSLI or vincristine to be injected (assuming 200 μl volume per mouse). Previously prepared VSLI was diluted to the appropriate concentration using phosphate buffer (made to clinical specifications). The exact volume to be injected into each mouse, to get the specific mg/kg dose, was determined by back calculating from the individual mouse weights and concentration of the VSLI or vincristine.

| Group | Drug Treatment | Schedule | Dose | Mice/group |
|---|---|---|---|---|
| A | VSLI | Q5dx3 | 1.5 mg/kg | 6 |
| B | VSLI | Q5dx3 | 1.0 mg/kg | 6 |
| C | Vincristine | Q5dx3 | 1.0 mg/kg | 6 |
| D | No Treatment | Q5dx3 | pH 7.5 | 6 |
| | | | PBS | |
| E | VSLI | Q3dx3 | 1.0 mg/kg | 6 |
| F | VSLI | Q3dx3 | 0.8 mg/kg | 6 |
| G | Vincristine | Q3dx3 | 0.8 mg/kg | 6 |
| H | No Treatment | Q3dx3 | pH 7.5 | 6 |
| | | | PBS | |

The growth of tumors was measured with calipers in the standard manner twice a week. The tumor volume (mm³) was calculated via the formula (L×W×H×π)/6 and plotted versus time. Mice were euthanized when their tumor volume reached a minimum of 1400 mm³.

### D. Example IV

### Efficacy of VSLI in Treating LX-1 Tumors in a Multiple Low Dose Treatment Schedule

This example illustrates a protocol that can be used to evaluate the efficacy of VSLI in treating LX-1 tumors in a multiple low dose treatment schedule.

LX-1 cells were obtained from the NCI and serially passaged in mice for 5 cycles before being used in this experiment. Tumors from passage animals were harvested when they reach 10-15 mm in diameter (300-600 mm³). The tumor was resected and cut in pieces of 2-3 mm³ (0.014-0.17 g). Tumor fragments of the appropriate size were implanted into the 60 mice using a 10 G trokar while mice were anesthetized with halothane. Sixty mice (60 NCR nu/nu mice, female, 20-23 g, 5-6 weeks old) were implanted with tumor, but up to 20% were excluded at time of treatment due to small tumor size.

When the average tumor size reached 100 mm³, the mice were randomized into groups (4 mice per group). Mice with very small tumors were excluded from the experiment. Twenty percent of mice implanted were excluded due to small tumor size, therefore, only 48 of the 60 mice implanted were treated. Mice were given seven treatments of VSLI or vincristine at the appropriate dose (based on their body weight) every three days or every seven days. Injections which fell on a Saturday were late Friday afternoon and injections which fell on a Sunday were given first thing Monday morning. Injections were given intravenously in the tail vein in approximately 200 μl.

When tumors reached an average size of 100 mm³ (11 days after implantation) mice were given their first treatment with either VSLI or vincristine. Subsequent treatments were given following the schedules listed below. Mice were weighed on the day of injection and the average weight of the mice in each group was used to calculate the concentration of VSLI or vincristine to be injected (assuming 200 μl volume per mouse). Previously prepared VSLI was diluted to the appropriate concentration using phosphate buffer (made to clinical specifications). The exact volume to be injected into each mouse, to get the specific mg/kg dose, was determined by back calculating from the individual mouse weights and concentration of the VSLI or vincristine.

| Group | Drug Treatment | Schedule | | Dose | Mice/group |
|---|---|---|---|---|---|
| A | VSLI | Q2dx7 | 0.1 × MTD | 0.2 mg/kg | 4 |
| B | Vincristine | Q2dx7 | 0.1 × MTD | 0.2 mg/kg | 4 |
| C | VSLI | Q2dx7 | 0.25 × MTD | 0.5 mg/kg | 4 |
| D | Vincristine | Q2dx7 | 0.25 × MTD | 0.5 mg/kg | 4 |
| E | No Treatment | Q2dx7 | | pH 7.5 | 4 |
| | | | | PBS | |
| F | VSLI | Q3dx7 | 0.1 × MTD | 0.2 mg/kg | 4 |
| G | Vincristine | Q3dx7 | 0.1 × MTD | 0.2 mg/kg | 4 |
| H | VSLI | Q3dx7 | 0.25 × MTD | 0.5 mg/kg | 4 |
| I | Vincristine | Q3dx7 | 0.25 × MTD | 0.5 mg/kg | 4 |
| J | No Treatment | Q3dx7 | | pH 7.5 | 4 |
| | | | | PBS | |

The growth of tumors was measured with calipers in the standard manner twice a week. The tumor volume (mM³) was calculated via the formula (L×W×H×π)/6 and plotted versus time. Mice were euthanized when their tumor volume reached a minimum of 1400 mm³.

### E. Example V

### Evaluation of Maximum Tolerated Dose of VSLI

This example illustrates a protocol that can be used to evaluate the maximum tolerated dose (MTD) of VSLI in various mouse stains.

The animals used in this study were as follows: 48 C57BL/6 mice, female, 20-23 g; 48 NCI nu/nu mice, female, 20-23 g; and 48 Balb/c mice, female, 20-23 g.

Mice were weighed on the day of injection and the average weight of the mice in each group was used to calculate the concentration of VSLI or vincristine to be injected (assuming 200 μl volume per mouse). Previously prepared VSLI was diluted to the appropriate concentration using phosphate buffer (made to clinical specifications). The exact volume to be injected into each mouse, to get the specific mg/kg dose, was determined by back calculating from the individual mouse weights and concentration of the VSLI or vincristine. Injections were administered intravenously in the tail vein in approximately 200 μl as set forth in the table below.

| | | | | | |
|---|---|---|---|---|---|
| Group | Drug Treatment | Schedule | Dose | Total Dose | Mice/group |

C57/BL6

| | | | | | |
|---|---|---|---|---|---|
| A | Vincristine | single | 1.0 mg/kg | 1.0 mg/kg | 4 |
| B | Vincristine | single | 1.5 mg/kg | 1.5 mg/kg | 4 |
| C | Vincristine | single | 2.0 mg/kg | 2.0 mg/kg | 4 |
| D | VSLI | single | 1.5 mg/kg | 1.5 mg/kg | 4 |
| E | VSLI | single | 2.0 mg/kg | 2.0 mg/kg | 4 |
| F | VSLI | single | 2.5 mg/kg | 2.5 mg/kg | 4 |
| G | Vincristine | Q1dx7 | 0.21 mg/kg | 1.5 mg/kg | 4 |
| H | Vincristine | Q3dx7 | 0.21 mg/kg | 1.5 mg/kg | 4 |
| I | Vincristine | Q7dx7 | 0.21 mg/kg | 1.5 mg/kg | 4 |
| J | VSLI | Q1dx7 | 0.29 mg/kg | 2.0 mg/kg | 4 |
| K | VSLI | Q3dx7 | 0.29 mg/kg | 2.0 mg/kg | 4 |
| L | VSLI | Q7dx7 | 0.29 mg/kg | 2.0 mg/kg | 4 |

NCI nu/nu

| A | Vincristine | single | 1.0 mg/kg | 1.0 mg/kg | 4 |
|---|---|---|---|---|---|
| B | Vincristine | single | 1.5 mg/kg | 1.5 mg/kg | 4 |
| C | Vincristine | single | 2.0 mg/kg | 2.0 mg/kg | 4 |
| D | VSLI | single | 1.5 mg/kg | 1.5 mg/kg | 4 |
| E | VSLI | single | 2.0 mg/kg | 2.0 mg/kg | 4 |
| F | VSLI | single | 2.5 mg/kg | 2.5 mg/kg | 4 |
| G | Vincristine | Q1dx7 | 0.21 mg/kg | 1.5 mg/kg | 4 |
| H | Vincristine | Q3dx7 | 0.21 mg/kg | 1.5 mg/kg | 4 |
| I | Vincristine | Q7dx7 | 0.21 mg/kg | 1.5 mg/kg | 4 |
| J | VSLI | Q1dx7 | 0.29 mg/kg | 2.0 mg/kg | 4 |
| K | VSLI | Q3dx7 | 0.29 mg/kg | 2.0 mg/kg | 4 |
| L | VSLI | Q7dx7 | 0.29 mg/kg | 2.0 mg/kg | 4 |
| Balb/c | | | | | 4 |
| A | Vincristine | single | 1.0 mg/kg | 1.0 mg/kg | 4 |
| B | Vincristine | single | 1.5 mg/kg | 1.5 mg/kg | 4 |
| C | Vincristine | single | 2.0 mg/kg | 2.0 mg/kg | 4 |
| D | VSLI | single | 1.5 mg/kg | 1.5 mg/kg | 4 |
| E | VSLI | single | 2.0 mg/kg | 2.0 mg/kg | 4 |
| F | VSLI | single | 2.5 mg/kg | 2.5 mg/kg | 4 |
| G | Vincristine | Q1dx7 | 0.21 mg/kg | 1.5 mg/kg | 4 |
| H | Vincristine | Q3dx7 | 0.21 mg/kg | 1.5 mg/kg | 4 |
| I | Vincristine | Q7dx7 | 0.21 mg/kg | 1.5 mg/kg | 4 |
| J | VSLI | Q1dx7 | 0.29 mg/kg | 2.0 mg/kg | 4 |
| K | VSLI | Q3dx7 | 0.29 mg/kg | 2.0 mg/kg | 4 |
| L | VSLI | Q7dx7 | 0.29 mg/kg | 2.0 mg/kg | 4 |

Mouse weights were recorded twice per week and the study continued until mouse weights decreased below 20% of original weight or mice became too ill; when severe toxicity was believed, mice were euthanized by CO₂ inhalation or cervical dislocation preceded by general anesthesia. Following euthanasia, mice were examined for the presence of tumor burden; any abnormal findings were noted.

### F. Example VI

### Efficacy of VSLI in Treating CT-26 Intrasplenic Tumors in a Multiple Low Dose Treatment Schedule

This example illustrates a protocol that can be used to evaluate the efficacy of VSLI in treating CT-26 intrasplenic tumors in a multiple low dose treatment schedule.

Colon 26, mouse metastatic colon carcinoma cells were maintained in culture in RPMI media supplemented with 10% FBS. Cells were harvested with 0.25% trypsin with two washes in PBS. On day 0, mice (40 Balb/c mice, female, 20-23 g) were anesthetized and 10,000 colon 26 cells (50 μl, in PBS) were injected with a 27-G needle into the exposed spleen parenchyma via a small incision. Ten-minutes following tumor cell administration, the spleen was removed and the incision closed with sutures.

Treatments were initated 24 hours after tumor cell implantation. The mice were randomized into groups (4 mice per group). Mice were given seven treatments of VSLI or vincristine at the appropriate dose (based on their body weight) every three days or every seven days. Injections were administered intravenously in the tail vein in approximately 200 μl.

Treatments were administered following the schedules listed in the table below. Mice were weighed on the day of injection and the average weight of the mice in each group was used to calculate the concentration of VSLI or vincristine to be injected (assuming 200 μl volume per mouse). Prepared VSLI was diluted to the appropriate concentration using phosphate buffer (made to clinical specifications). The exact volume to be injected into each mouse, the specific mg/kg dose, was determined from the individual mouse weights and concentration of the VSLI or vincristine.

| Group | Drug Treatment | Schedule | | Dose | Mice/group |
|---|---|---|---|---|---|
| A | VSLI | Q2dx7 | 0.1 × MTD | 0.2 mg/kg | 4 |
| B | Vincristine | Q2dx7 | 0.1 × MTD | 0.2 mg/kg | 4 |
| C | VSLI | Q2dx7 | 0.25 × MTD | 0.5 mg/kg | 4 |
| D | Vincristine | Q2dx7 | 0.25 × MTD | 0.5 mg/kg | 4 |
| E | No Treatment | Q2dx7 | | pH 7.5 | 4 |
| | | | | PBS | |
| F | VSLI | Q3dx7 | 0.1 × MTD | 0.2 mg/kg | 4 |
| G | Vincristine | Q3dx7 | 0.1 × MTD | 0.2 mg/kg | 4 |
| H | VSLI | Q3dx7 | 0.25 × MTD | 0.5 mg/kg | 4 |
| I | Vincristine | Q3dx7 | 0.25 × MTD | 0.5 mg/kg | 4 |
| J | No Treatment | Q3dx7 | | pH 7.5 | 4 |
| | | | | PBS | |

Mouse weights were recorded twice per week and the study continued until mouse weights decreased below 20% of original weight or mice became too ill; when severe toxicity was believed, mice were euthanized by CO₂ inhalation or cervical dislocation preceded by general anesthesia. Following euthanasia, mice were examined for the presence of tumor burden; any abnormal findings were noted.

### G. Example VII

### Evaluation of the Efficacy of Varying Drug-to-Lipid Ratios of VSLI in the LX-1, CT-26 and Lewis Lung Tumor Models

This example illustrates a protocol for evaluating the efficacy of varying drug-to-lipid ratios of VSLI in various tumor models.

The various tumor models were prepared as follows:

LX-1 cells were obtained from the NCI and serially passaged in mice for 5 cycles before being used in this experiment. Tumors from passage animals were harvested when they reach 10-15 mm in diameter (300-600 mm³). The tumor was resected and cut in pieces of 2-3 mm³. Tumor fragments of the appropriate size were implanted into the mice (20 NCI nu/nu mice, female, 20-23 g) using a 10G trokar while mice were anesthetized with halothane. Mice were implanted with tumor, but up to 20% were excluded at time of treatment due to small tumor size.

Colon 26, mouse metastatic colon carcinoma cells were maintained in culture in RPMI media supplemented with 10% FBS. Cells were harvested with 0.25% trypsin with two washes in PBS. On day 0, mice (20 Balb/c mice, female, 20-23 g) were anesthetized and 10,000 colon 26 cells (50 μl, in PBS) were injected with a 27-G needle into the exposed spleen parenchyma via a small incision. Ten-minutes following tumor cell administration, the spleen was removed and the incision closed with sutures.

Lewis Lung Carcinoma cells were maintained in DMEM media supplemented with 10% FBS. Cells were harvested by rinsing with PBS and then dislodged from the tissue culture flask by agitating the PBS with a pipette. On day 0, 10⁶ cells (50 μL, in HBSS) were injected with a 27-G½ needle into the right lateral posterior flank of the mice (20 C57BL/6 mice, female, 18-21 g) while they were anesthetized with halothane.

Mice were weighed on the day of injection and the average weight of the mice in each group was used to calculate the concentration of VSLI or vincristine to be injected (assuming 200 μl volume per mouse). Prepared VSLI was diluted to the appropriate concentration using phosphate buffer (made to clinical specifications). The exact volume to be injected into each mouse, the specific mg/kg dose, was determined from the individual mouse weights and concentration of the VSLI or vincristine.

Injections were administered intravenously in the tail vein in approximately 200 μl as indicated in the table below. For solid tumor models, Lewis Lung and LX-1, treatments were initiated when tumors reach a size of 20 to 70 mm³. For the CT-26 intrasplenic study, mice were treated 24-hours after tumor cell implantation.

| | | | | | |
|---|---|---|---|---|---|
| | Drug Treatment | | | | |
| Group | (D/L) | Schedule | Dose | Total Dose | Mice/group |

LX-1

| | | | | | |
|---|---|---|---|---|---|
| A | Vincristine | Q3dx7 | 0.21 mg/kg | 1.5 mg/kg | 5 |
| B | VSLI (0.1) | Q3dx7 | 0.29 mg/kg | 2.0 mg/kg | 5 |
| C | VSLI (0.05) | Q3dx7 | 0.29 mg/kg | 2.0 mg/kg | 5 |
| D | VSLI (0.01) | Q3dx7 | 0.29 mg/kg | 2.0 mg/kg | 5 |

CT-26

| | | | | | |
|---|---|---|---|---|---|
| A | Vincristine | Q3dx7 | 0.21 mg/kg | 1.5 mg/kg | 5 |
| B | VSLI (0.1) | Q3dx7 | 0.29 mg/kg | 2.0 mg/kg | 5 |
| C | VSLI (0.05) | Q3dx7 | 0.29 mg/kg | 2.0 mg/kg | 5 |
| D | VSLI (0.01) | Q3dx7 | 0.29 mg/kg | 2.0 mg/kg | 5 |

Lewis Lung

| | | | | | |
|---|---|---|---|---|---|
| A | Vincristine | Q3dx7 | 0.21 mg/kg | 1.5 mg/kg | 5 |
| B | VSLI (0.1) | Q3dx7 | 0.29 mg/kg | 2.0 mg/kg | 5 |
| C | VSLI (0.05) | Q3dx7 | 0.29 mg/kg | 2.0 mg/kg | 5 |
| D | VSLI (0.01) | Q3dx7 | 0.29 mg/kg | 2.0 mg/kg | 5 |

The growth of tumors was measured with calipers in the standard manner twice a week. The tumor volume (mm³) was calculated via the formula (L×W×H×π)/6 and plotted versus time. Mice were euthanized when their tumor volume reached a minimum of 1400 mm³.

Mouse weights were recorded twice per week and the study continued until mouse weights decreased below 20% of original weight or mice became too ill; when severe toxicity was believed, mice were euthanized by CO₂ inhalation or cervical dislocation preceded by general anesthesia. Following euthanasia, mice were examined for the presence of tumor burden; any abnormal findings were noted.

### H. Example VIII

### Evaluation of the Efficacy of VSLI in Treating Drug Resistant Lewis Lung Tumor in a Multiple Low Dose Treatment Schedule

Lewis Lung Carcinoma cells were maintained in DMEM media supplemented with 10% FBS. Cells were harvested by rinsing with PBS and then dislodged from the tissue culture flask by agitating the PBS with a pipette. On day 0, 10⁶ cells (50 μL, in HBSS) were injected with a 27-G½ needle into the right lateral posterior flank of the mice (20 C57BL/6 mice, female, 18-21 g) while they were anesthetized with halothane.

Treatments were initated when tumors reached a size 100-200 mm³. The mice were randomized into groups (4 mice per group). Mice were given seven treatments of VSLI or vincristine at the appropriate dose (based on their body weight) every three days or every seven days. Injections were administered intravenously in the tail vein in approximately 200 μl.

Treatments were administered following the schedules listed in the table below. Mice were weighed on the day of injection and the average weight of the mice in each group was used to calculate the concentration of VSLI or vincristine to be injected (assuming 200 μl volume per mouse). Prepared VSLI was diluted to the appropriate concentration using phosphate buffer (made to clinical specifications). The exact volume to be injected into each mouse, the specific mg/kg dose, was determined from the individual mouse weights and concentration of the VSLI or vincristine.

| Group | Drug Treatment | Schedule | | Dose | Mice/group |
|---|---|---|---|---|---|
| A | VSLI | Q2dx7 | 0.1 × MTD | 0.2 mg/kg | 4 |
| B | Vincristine | Q2dx7 | 0.1 × MTD | 0.2 mg/kg | 4 |
| C | VSLI | Q2dx7 | 0.25 × MTD | 0.5 mg/kg | 4 |
| D | Vincristine | Q2dx7 | 0.25 × MTD | 0.5 mg/kg | 4 |
| E | No Treatment | Q2dx7 | | pH 7.5 | 4 |
| | | | | PBS | |
| F | VSLI | Q3dx7 | 0.1 × MTD | 0.2 mg/kg | 4 |
| G | Vincristine | Q3dx7 | 0.1 × MTD | 0.2 mg/kg | 4 |
| H | VSLI | Q3dx7 | 0.25 × MTD | 0.5 mg/kg | 4 |
| I | Vincristine | Q3dx7 | 0.25 × MTD | 0.5 mg/kg | 4 |
| J | No Treatment | Q3dx7 | | pH 7.5 | 4 |
| | | | | PBS | |

The growth of tumors was measured with calipers in the standard manner twice a week. The tumor volume (mM³) was calculated via the formula (L×W×H×π)/6 and plotted versus time. Mice were euthanized when their tumor volume reached a minimum of 1400 mm³.

Mouse weights were recorded twice per week and the study continued until mouse weights decreased below 20% of original weight or mice became too ill; when severe toxicity was believed, mice were euthanized by CO₂ inhalation or cervical dislocation preceded by general anesthesia. Following euthanasia, mice were examined for the presence of tumor burden; any abnormal findings were noted.

### I. Example IX

### In Vitro and In Vivo Selection of Vincristine Resistant LLC Cells

A. In Vitro Selection of Vincristine Resistant LLC Cells

A vincristine (VCR) resistant Lewis Lung Carcinoma (LLC) cell line will be generated and designated LLC/VCR. The methods used to generate a LLC/VCR cell line are similar to those used to select for cells able to grow in the presence of a selective agent (see, e.g., Freshney et al., Culture of Animal Cells, A Manual of Basic Technique, Third Edition, Wiley-Liss, New York (1994), pages 172-173). Briefly, cells are exposed to gradually increasing concentrations of a selective agent (e.g., a chemotherapeutic, a cytotoxic compound, etc.) over a prolonged period of time until cells are selected that are able to grow at a desired concentration of the selected agent.

To generate the LLC/VCR cells, LLC cells will be selected for their ability to grow at the same rate as a control group of LLC cells in the absence of VCR. Initially, the LLC cells will be selected in the presence of low (e.g., 4 nM) concentrations of vincristine. Once resistant cells are obtained, those cells will be subjected to increasing concentrations of vincristine until the desired concentration of resistance (e.g., 600 nM) is reached. For instance, cells that can grow in the presence of 600 nM vincristine are considered LLC/VCR cells.

Cells are plated and counted using standard cell culture methods. Cell viability can be determined using methods known in the art, e.g., by using a hemocytometer and the trypan blue dye exclusion assay or an alamarblue assay (Alamar Bio-Sciences, Sacramento, Calif.). The determination of the optimal cell number per dish and the optimal exposure time to a selective agent can be carried out using methods well within the purview of one of skill in the art. In the following protocol, Vincristine sulfate (Vincasar) is the selective agent:

In vitro Selection Protocol

1. Expose LLC cells in triplicate to vincristine concentrations of 0, 4, 6, 8, 16 and 32 nM in cell culture media.

2. Passage the cells 6-8 times until their growth rate approaches that of control (0 nM vincristine). Determine the concentration of drug “Y” (i.e., vincristine sulfate) that gives the highest number of cell-count in the shortest time.

3. Expose these cells to twice “Y” in two flasks.

4. Plate another two flasks of cells and expose them to drug at “Y” after 24 hours of incubation.

5. Passage both groups (in step 3 and step 4) for 6-8 weeks until growth rate approaches that of control.

6. Increase the drug concentration by 2-fold and repeat steps 2-5.

The selection is completed when the growth rate of selected cells incubated in 600 nM (˜7 cycles of 6-8 passages each) is comparable to that of control. The generated cell line is designated as LLC/VCR. The LLC/VCR should be maintained in the presence of about 300 nM or greater vincristine.

### B. In Vivo Selection of Vincristine Resistant LLC Cells

Typically 5 to 6 weeks old female C57BL6/J mice at 23-28 grams are used in this procedure. These mice are commonly used for propagating Lewis Lung Carcinoma and the strain is recommended by American Type Culture Collection. In the first phase, the MTD will be determined for vincristine in C57BL6/J mice by intravenous injection. MTD is the highest dose of drug that can be administered to a group of mice with weight lost less than or equal to 20%. In the second phase, LLC/VCR cells will be injected and subjected to VCR or VSLI at the MTD Dose. Cells obtained from the resistant tumors will be exposed to vincristine in vitro to confirm the resistance phenotype. Those cells found to have the resistance phenotype are considered VSLI or VCR resistant.

First phase—Determination of the MTD Dose

Nine groups of mice will be injected with LLC cells and subsequently injected with vincristine (VCR) or Vincristine Sulfate Liposomes Injection (VSLI) (vincristine sulfate (Vincasar) in a sphingomyelin/cholesterol liposome) (see, Table 1).

TABLE 1

| Group | Cells injected | Agent | Dosage |
|---|---|---|---|
| Group 1 | LLC | PBS | Not |
| | | | applicable |
| Group 2 | LLC | VCR | 1.5 mg/kg |
| Group 3 | LLC | VCR | 2 mg/kg |
| Group 4 | LLC | VCR | 2.5 mg/kg |
| Group 5 | LLC | VCR | 3 mg/kg |
| Group 6 | LLC | VSLI | 3 mg/kg |
| Group 7 | LLC | VSLI | 4 mg/kg |
| Group 8 | LLC | VSLI | 4.8 mg/kg |
| Group 9 | LLC | VSLI | 5.3 mg/kg |

Each mouse is inoculated subcutaneously with 10⁶ of LLC cells in 100 uL in the right lateral posterior flank. After the tumors reach 100-200 mm³ (2-4 days after inoculation of cells) the mice are injected intravenously with 200 uL of VCR at 1.5, 2, 2.5 and 3 mg/kg or VSLI at 3, 4, 4.8 and 5.3 mg/kg at single dose. A group of 4 mice will be injected with 200 uL of PBS as a control. The mouse weights will be recorded everyday until the weights stabilize (˜1 week). The MTD is the dose at which mice lose 20% of their original weight.

Second Phase

In this phase mice will be injected with LLC or LLC/VCR cells and then injected with VCR or VSLI at the MTD determined in the First Phase. Each mouse is inoculated with 10⁶ of LLC or LLC/VCR cells in subcutaneously in the right lateral posterior flank as set out in Table 2.

TABLE 2

| Group | Cells Injected | Agent Injected |
|---|---|---|
| Group A | LLC | PBS |
| Group B | LLC/VCR | PBS |
| Group C | LLC | VCR |
| Group D | LLC/VCR | VCR |
| Group E | LLC | VSLI |
| Group F | LLC/VCR | VSLI |

The mice in groups B and D will be injected with vincristine at the MTD (as determined in the first phase). A single dose will be administered when tumors reach 100-200 mm³ (2-4 days). Mice in groups A and C will be injected with of PBS. The route of drug administration is via an intravenous tail vein.

Tumor size and mice weights are monitored daily. The development of a LLC/VCR cell line is completed when the growth rates of tumors in group A and D are comparable (5% difference).

In the case where there is a difference between growth rates of tumors in group A and group D, the selection process is repeated as in the first phase for 3 cycles followed by the second phase.

Post-experimental Recommendations

Prior to using LLC/VCR for any experiment, one should test for level of drug resistance of LLC/VCR. Mice can be treated 2-4 days after inoculation with LLC/VCR.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating or preventing a disease or condition with an angiogenic component in a mammal, said method comprising administering to said patient a pharmaceutical composition comprising a liposome-encapsulated chemotherapeutic agent, wherein said pharmaceutical composition is administered to said mammal at an average frequency of at least once every 7 days for a total period of at least 6 weeks.

2. The method of claim 1, wherein said disease or condition is cancer.

3. The method of claim 1, wherein said disease or condition is multiple myeloma.

4. The method of claim 1, wherein said chemotherapeutic agent is an alkaloid.

5. The method of claim 4, wherein said alkaloid is a vinca alkaloid.

6. The method of claim 5, wherein said vinca alkaloid is vincristine.

7. The method of claim 6, wherein said vincristine is administered to said mammal at a dosage of less than 0.5 mg/m².

8. The method of claim 6, wherein said vincristine is administered to said mammal at a dosage of les than 0.1 mg/m².

9. The method of claim 5, wherein said alkaloid is vinorelbine or vinblastine.

10. The method of claim 1, wherein said chemotherapeutic agent is a camptothecin or a camptothecin analog.

11. The method of claim 10, wherein said camptothecin is topotecan.

12. The method of claim 1, wherein said composition is administered to said mammal for a total period of at least 10 weeks.

13. The method of claim 1, wherein said composition is administered to said mammal for a period of longer than 10 weeks.

14. The method of claim 1, further comprising co-administering an angiogenesis inhibitor to said mammal.

15. The method of claim 14, wherein said angiogenesis inhibitor is selected from the group consisting of thrombospondin, internal fragments of thrombospondin, angiostatin, endostatin, vasostatin, vascular endothelial growth factor inhibitor (VEGI), fragment of platelet factor 4 (PP4), derivative of prolactin, restin, proliferin-related protein (PRP), SPARC cleavage product, osteopontin cleavage product, interferon α, interferon β, meth 1, meth I, angiopoietin-2, anti-thrombin III fragment, COL-3, squalamine, combretastatin, PTK787/ZK2284, CAI, PIK787/2K22584, CGS-27023A, TNP-470, thalidomide, SU5416, vitaxin, IL-12, EMD121974, marimastat, AG3340, neovastat/AE941, anti-VEGF Ab, and IM862.

16. The method of claim 1, wherein said liposome comprises sphingomyelin.

17. The method of claim 16, wherein said liposome further comprises cholesterol.

18. The method of claim 1, wherein said liposome comprises a PEG-lipid.

19. The method of claim 1, wherein said liposome comprises an ATTA-lipid.

20. The method of claim 1, wherein said pharmaceutical composition is administered to said patient following a primary cancer treatment, and said method is used to delay or prevent relapse of said cancer in said patient.

21. The method of claim 3, wherein said pharmaceutical composition is administered to said patient following a primary cancer treatment, and said method is used to delay or prevent relapse of said cancer in said patient.

22. The method of claim 1, wherein said pharmaceutical composition is administered to said patient following a primary cancer treatment, and said method is used to prevent metastasis of said cancer in said patient.

23. The method of claim 2, wherein said cancer comprises a primary tumor that is resistant to said chemotherapeutic alkaloid.

24. The method of claim 1, further comprising co-administering to said patient an oligonucleotide agent.

25. The method of claim 1, wherein said disease is selected from the group consisting of age-related macular degeneration, diabetic retinopathy, rubeotic glaucoma, interstitial keratitis, retinopathy of prematurity, corneal graft failure, psoriasis, atherosclerosis, restenosis, chronic inflammation, rheumatoid arthritis, vasculopathies including hemangiomas and systemic vasculitis.

26. A method of treating a vincristine-resistant tumor in a mammal, said method comprising administering to said mammal a pharmaceutical composition comprising liposome-encapsulated vincristine, wherein said pharmaceutical composition is administered to said mammal at an average frequency of at least once every 7 days for a total period of at least 6 weeks.

27. A dosage form of liposome-encapsulated vincristine, said dosage form comprising less than 0.5 mg/m² of vincristine per dose.

28. The dosage form of claim 27, wherein said vincristine is present at less than about 0.2 mg/m² per dose.

29. The dosage form of claim 27, wherein said vincristine is present at less than about 0.1 mg/m² per dose.

30. The dosage form of claim 27, wherein said liposome comprises sphingomyelin.

31. The method of claim 30, wherein said liposome further comprises cholesterol.

32. The method of claim 27, wherein said liposome comprises a PEG-lipid.

33. The method of claim 27, wherein said liposome comprises an ATTA-lipid.
